(19) United States
(12) Patent Application Publication
(10) Pub. No.: US 2007/0078413 A1
(43) Pub. Date: Apr. 5, 2007

(54) Medical device having a lubricant

(76) Inventor: Eric B. Stenzel, Tuam (IE)

Correspondence Address: COOLEY GODWARD KRONISH LLP;ATTN: PATENT GROUP, Suite 500, 1200 - 19th Street, NW, WASHINGTON, DC, 20036-2402, US

(21) Appl. No.: 11/210,724

(22) Filed: Aug. 25, 2005

(51) Int. Cl. A61M 25/00; A61M 5/00; A61M 31/00; A61M 5/32

(52) U.S. Cl. 604/265; 604/172; 604/509

(57) ABSTRACT

A medical device includes a member, a coating, and a lubricant. In one embodiment, the coating includes a therapeutic agent. In one embodiment, the coating is disposed on at least a portion of the body and the lubricant is disposed on at least a portion of the coating. In one embodiment, the lubricant is formulated to provide an effective degree of lubricity between the coating and at least one of a surface of a package configured to receive at least a portion of the medical device, another portion of the medical device, a coating of another medical device, and an uncoated portion of another medical device. In one embodiment, the lubricant is soluble in at least one of water and a bodily fluid of a mammal. In one embodiment, the coating is formulated to release from the member when the medical device is placed within a body of a patient.

### BACKGROUND

The present invention relates generally to a medical device and more particularly to a coated medical device, such as a stent that includes a coated surface.

Some known medical devices are configured to be implanted into a body of a patient and include coatings. For example, some known stents include a coating that has a therapeutic agent disposed therein. The coatings of such known medical devices, however, may have tacky or sticky surfaces. Processing and/or handling of such known medical devices may be made difficult because of the tacky surface. For example, such a medical device may stick to the medical device's packaging and may thus be damaged upon removal of the medical device from the packaging. Additionally, such known medical devices may stick to each other and may be damaged upon separation of the medical devices.

Accordingly, there is a need for a coated medical device that does not include a tacky or sticky surface.

### SUMMARY OF THE INVENTION

A medical device includes a member, a coating, and a lubricant. In one embodiment, the coating includes a therapeutic agent. In one embodiment, the coating is disposed on at least a portion of the body and the lubricant is disposed on at least a portion of the coating. In one embodiment, the lubricant is formulated to provide an effective degree of lubricity between the coating and at least one of a surface of a package configured to receive at least a portion of the medical device, another portion of the medical device, a coating of another similar medical device, and an uncoated portion of another medical device. In one embodiment, the lubricant is soluble in at least one of water and a bodily fluid of a mammal. In one embodiment, the coating is formulated to release from the member when the medical device is placed within a body of a patient.

### BRIEF DESCRIPTION OF THE DRAWINGS

FIG. 1 is a schematic illustration of a medical device according to the disclosed invention.

FIG. 2 is a perspective view of a medical device according to an embodiment of the disclosed invention.

FIG. 2A is a perspective view of the medical device of FIG. 2 being inserted into a packaging.

FIG. 2B is a perspective view of the medical device of FIG. 2 disposed within a packaging.

FIG. 3 is a side view of the medical device of FIG. 2.

FIG. 4 is a cross-sectional view of the medical device of FIG. 2 taken along line 4-4 of FIG. 3.

FIG. 5 is a cross-sectional view of a medical device according to another embodiment of the disclosed invention.

FIGS. 6-8 are cross-sectional views of medical devices according to other embodiments of the disclosed invention.

### DETAILED DESCRIPTION

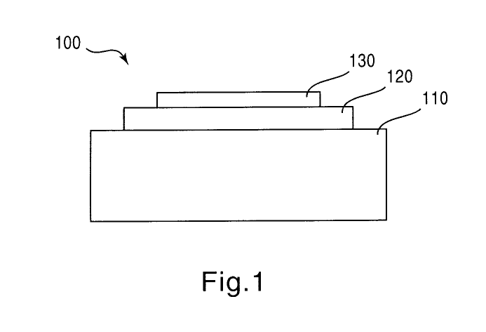

FIG. 1 is a schematic illustration of a medical device 100 according to an embodiment of the disclosed invention. The medical device 100 includes a member 110, a coating 120, and a lubricant 130. The medical device 100 is configured to be inserted, placed, or otherwise disposed within a body of a mammalian or human patient.

J The member 110 can be any shape. For example, the body 110 can be spherical, tubular, cubic, or a mixture of shapes.

The member 110 may be formed from any material or materials known in the art to be used in constructing medical devices configured to be inserted, placed or otherwise disposed within a body of a mammal or human patient. One subset of biocompatible materials best suited for the member 110 may exhibit at least some of the following characteristics: high tensile strength, excellent biocompatibility and biodurability, excellent radiopacity or flouroscopic visibility, availability in varying durometers, and a low resistance to passage. For example, in one embodiment, the member 110 is formed from a polymeric material. In another embodiment, the member 110 is formed from a metal.

The coating 120 of the medical device 100 is disposed on at least a portion of the member 110. Similarly, the lubricant 130 of the medical device 100 is disposed on at least a portion of the coating 120. In another embodiment, the coating and the lubricant are each disposed on at least a portion of the member.

In one embodiment, the coating 120 is sticky or tacky. Accordingly, in such an embodiment, a surface of the portion of the medical device that includes the coating 120 is sticky or tacky.

In one embodiment, the coating 120 includes a therapeutic agent. The therapeutic agent is formulated to treat a mammalian or human patient.

As used herein, the term “therapeutic agent,” and similar terms, includes, but is not limited to, any therapeutic agent or active material, such as drugs, genetic materials, and biological materials. Suitable genetic materials include, but are not limited to, DNA or RNA, such as, without limitation, DNA/RNA encoding a useful protein, DNA/RNA intended to be inserted into a human body including viral vectors and non-viral vectors, and RNAi (RNA interfering sequences). Suitable viral vectors include, for example, adenoviruses, gutted adenoviruses, adeno-associated viruses, retroviruses, alpha viruses (Semliki Forest, Sindbis, etc.), lentiviruses, herpes simplex viruses, ex vivo modified and unmodified cells (e.g., stem cells, fibroblasts, myoblasts, satellite cells, pericytes, cardiomyocytes, skeletal myocytes, macrophage), replication competent viruses (e.g., ONYX-015), and hybrid vectors. Suitable non-viral vectors include, for example, artificial chromosomes and mini-chromosomes, plasmid DNA vectors (e.g., PCOR), cationic polymers (e.g., polyethyleneimine, polyethyleneimine (PEI)) graft copolymers (e.g., polyether-PEI and polyethylene oxide-PEI), neutral polymers PVP, SP1017 (SUPRATEK), lipids or lipoplexes, nanoparticles and microparticles with and without targeting sequences such as the protein transduction domain (PTD).

Suitable biological materials include, but are not limited to, cells, yeasts, bacteria, proteins, peptides, cytokines, and hormones. Examples of suitable peptides and proteins include growth factors (e.g., FGF, FGF-1, FGF-2, VEGF, Endothelial Mitogenic Growth Factors, and epidermal growth factors, transforming growth factor α and β, platelet derived endothelial growth factor, platelet derived growth factor, tumor necrosis factor α, hepatocyte growth factor and insulin-like growth factor), transcription factors, proteinkinases, CDK inhibitors, thymidine kinase, and bone morphogenic proteins (BMP's), such as BMP-2, BMP-3, BMP-4, BMP-5, BMP-6 (Vgr-1), BMP-7 (OP-1), BMP-8. BMP-9, BMP-10, BMP-11, BMP-12, BMP-13, BMP-14, BMP-15, and BMP-16. Currently preferred BMP's are BMP-2, BMP-3, BMP-4, BMP-5, BMP-6, and BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at a desired site. The delivery media can be formulated as needed to maintain cell function and viability. Cells include, for example, whole bone marrow, bone marrow derived mono-nuclear cells, progenitor cells (e.g., endothelial progentitor cells), stem cells (e.g., mesenchymal, hematopoietic, neuronal), pluripotent stem cells, fibroblasts, macrophage, and satellite cells.

The term “therapeutic agent” and similar terms also includes non-genetic agents, such as: anti-thrombogenic agents such as heparin, heparin derivatives, urokinase, and PPack (dextrophenylalanine proline arginine chloromethylketone); anti-proliferative agents such as enoxaprin, angiopeptin, or monoclonal antibodies capable of blocking smooth muscle cell proliferation, hirudin, and acetylsalicylic acid, amlodipine and doxazosin; anti-inflammatory agents such as glucocorticoids, betamethasone, dexamethasone, prednisolone, corticosterone, budesonide, estrogen, sulfasalazine, and mesalamine; antineoplastic/antiproliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, taxol and its analogs or derivatives; anesthetic agents such as lidocaine, bupivacaine, and ropivacaine; anti-coagulants such as D-Phe-Pro-Arg chloromethyl keton, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors and tick antiplatelet peptides; vascular cell growth promotors such as growth factors, Vascular Endothelial Growth Factors (VEGF, all types including VEGF-2), growth factor receptors, transcriptional activators, Insulin Growth Factor (IGF), Hepatocyte Growth Factor (HGF), and translational promotors; vascular cell growth inhibitors such as antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents, vasodilating agents, and agents which interfere with endogenous vasoactive mechanisms; anti-oxidants, such as probucol; antibiotic agents, such as penicillin, cefoxitin, oxacillin, tobranycin; angiogenic substances, such as acidic and basic fibrobrast growth factors, estrogen including estradiol (E2), estriol (E3) and 17-Beta Estradiol; and drugs for heart failure, such as digoxin, beta-blockers, angiotensin-converting enzyme (ACE) inhibitors including captopril and enalopril.

Preferred therapeutic materials include anti-proliferative drugs such as steroids, vitamins, and restenosis-inhibiting agents such as cladribine. Preferred restenosis-inhibiting agents include microtubule stabilizing agents such as Taxol, paclitaxel, paclitaxel analogues, derivatives, and mixtures thereof. For example, derivatives suitable for use in the present invention include 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl) glutamine, and 2′-O-ester with N-(dimethylaminoethyl) glutamide hydrochloride salt. Other preferred therapeutic materials include nitroglycerin, nitrous oxides, antibiotics, aspirins, digitalis, and glycosides.

In another embodiment, the coating 120 includes a therapeutic agent and a carrier. The therapeutic agent is formulated to treat a mammalian or human patient. The carrier is configured to help the therapeutic agent adhere to the member 110.

The term “carrier,” as used herein, refers to a diluent, adjuvant (e.g., Freund's adjuvant (complete and incomplete) or, more preferably, MF59C.1 adjuvant available from Chiron, Emeryville, Calif.), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Other examples of suitable pharmaceutical vehicles are described in “Remington: the Science and Practice of Pharmacy”, 20th ed., by Mack Publishing Co. 2000.

In one embodiment, the coating 120, including the therapeutic agent, or at least a portion of the coating 120 is configured to release from the member 110 when the medical device 100 is inserted, placed or otherwise disposed within a body of a patient. For example, in one embodiment, the coating 120 is formulated to dissolve, or otherwise to release the therapeutic agent, when placed in contact with a bodily fluid, such as blood or urine. Accordingly, when the medical device 100 is placed within a body of a patient, the therapeutic agent, alone or with coating 120, is released from the body 110 to travel within and treat the body of the patient.

The lubricant 130 is formulated to provide lubricity between the coating 120 and surfaces of other objects. In one embodiment, the lubricant 130 is formulated to provide an effective degree of lubricity between the coating 120 and a surface of a package. For example, the lubricant 130 is formulated provide an effective degree of lubricity between the coating 120 and a surface of a package that is configured to receive at least a portion of the medical device 100. In another embodiment, the lubricant is formulated to provide an effective degree of lubricity between the coating and a coating of another medical device (whether the same as, or different from, medical device 100). In yet another embodiment, the lubricant is formulated to provide an effective degree of lubricity between the coating and a surface of a package and between the coating and a coating of another medical device. In yet another embodiment, the lubricant is formulated to provide an effective degree of lubricity between the coating and another medical device, such as a balloon catheter. In yet another embodiment, the lubricant is formulated to provide an effective degree of lubricity between the coating and another portion of the medical device.

The term “effective degree” is used herein to mean, for example, a sufficient amount. Accordingly, an “effective degree” of lubricity between a first object and a second object means a sufficient amount of a smooth or slippery quality between the first object and the second object. Thus, the first object may be in contact with the second object and may move with respect to the second object without damaging any surface of the first object or the second object and without requiring application of such force to separate the first object from the second object as would cause damage (structural or cosmetic) to either object. In some instances the damage cannot be completely eliminated but the use of a lubricant can substantially reduce the damage to an acceptable level as compared to a device that does not include the lubricant.

In one embodiment, the lubricant 130 is formulated to be soluble in water. In another embodiment, the lubricant is formulated to be soluble in at least one bodily fluid, such as blood or urine. In yet another embodiment, the lubricant is formulated to be soluble in water and in at least one bodily fluid, such as blood or urine. In such an embodiment, the lubricant is formulated to stick to the coating and/or the member until the medical device is placed in contact with water and/or the at least one bodily fluid.

In one embodiment, the lubricant 130 is compatible with the coating 120 and the therapeutic agent. In other words, the lubricant 130 may be applied to the medical device 100 and/or the coating 120 without functionally damaging the coating 120 or the therapeutic agent.

In one embodiment, the lubricant 130 is formulated as a soluble powder. In an other embodiment, the lubricant 130 is formulated as a soluble biocompatible powder. For example, in one embodiment, the lubricant 130 is a soluble biocompatible powder such as potassium chloride, another salt, dried heparin, Mannitol, or ReoPro® (Abciximab).

The term “powder” is used herein to mean any type of solid particles. For example, a powder may be granules, pellets, or any other type of particles. In one embodiment, the powder may be formed by crushing, grinding, or otherwise attriting solid matter.

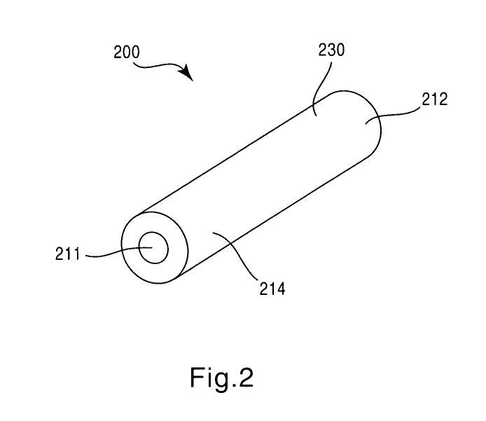

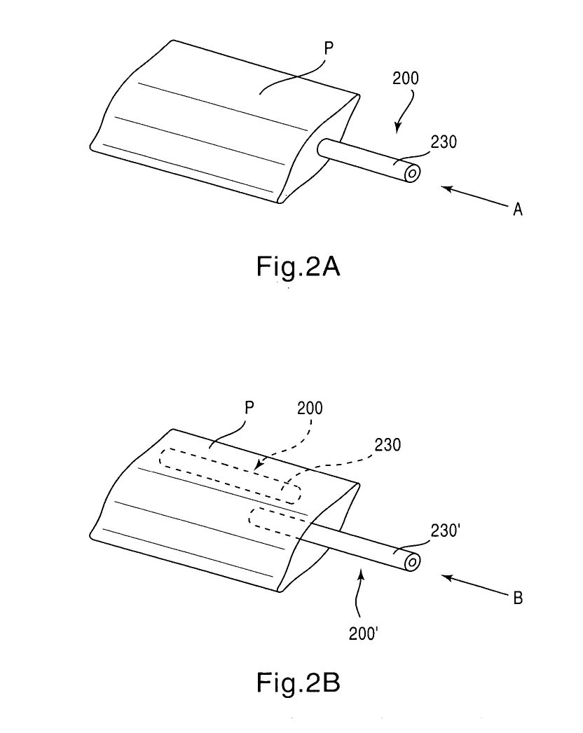

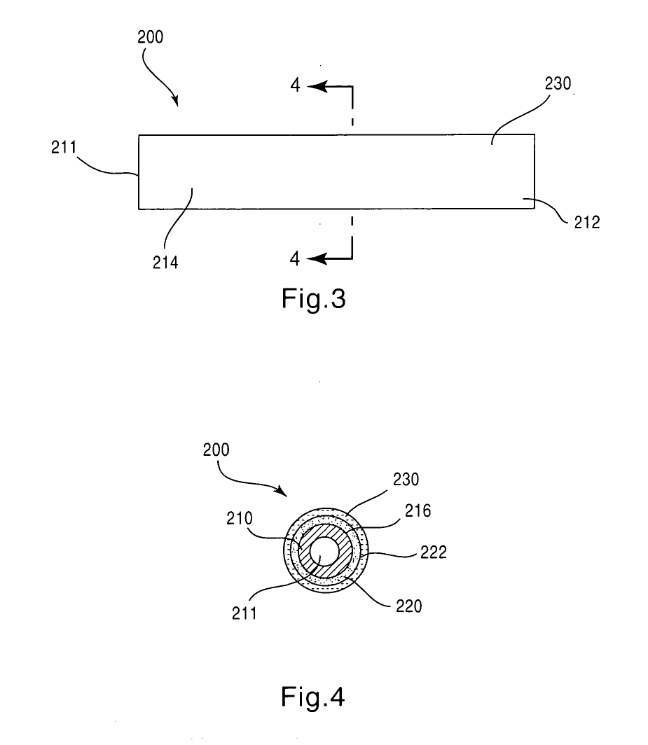

FIGS. 2, 2A, 2B, 3, and 4 illustrate another medical device 200 according to an embodiment of the disclosed invention. The medical device 200 includes a member 210, a coating 220, and a lubricant 230. The medical device 200 is configured to be placed or otherwise disposed within a body of a mammal or human patient.

The member 210 is a tubular member, such as a coronary or urinary stent, and is configured to be placed or otherwise disposed within a lumen of the human patient, such as a blood vessel or a ureter. The member 210 defines a lumen 211 and includes a first end portion 212 and a second end portion 214. The lumen 211 extends from the first end portion 212 to the second end portion 214.

In the illustrated embodiment, the member 210 is formed from a polymeric material. In another embodiment, the member is formed from a metal.

The coating 220 of the medical device 200 is disposed on a portion of the member 210. Similarly, the lubricant 230 of the medical device 200 is disposed on a portion of the coating 220. As best illustrated in FIG. 4, the coating 220 is disposed on the entirety of an outer surface 216 of the member 210, and the lubricant 230 is disposed on the entirety an outer surface 222 of the coating 220. However, the lubricant 230 need not cover the entirety of outer surface 222, and the coating 220 need not cover the entirety of outer surface 216.

The coating 220 includes a therapeutic agent. In the illustrated embodiment, the coating 220 includes a therapeutic agent that is formulated to treat a human patient. For example, in one embodiment, the therapeutic agent is one of the agents identified above.

In one embodiment, the coating 220 and/or the therapeutic agent is configured to release from the member 210 when the medical device 200 is inserted, placed or otherwise disposed within a body of a patient. In the illustrated embodiment, the coating 220 is formulated to dissolve, or at least partially dissolve, when placed in contact with a bodily fluid such as blood or urine. Accordingly, when the medical device 200 is placed within a body of a patient, the coating 220 and/or the therapeutic agent is released from the body 210 to travel within and treat the body of the patient. Alternatively, the therapeutic agent may be released from the coating, such as by migrating through pores in the coating, dissolving from cavities formed in the coating, etc.

The lubricant 230 is formulated to provide lubricity between the coating 220 and surfaces of other objects. As illustrated in FIGS. 2A and 2B, in one embodiment, the lubricant 230 is formulated to provide an effective degree of lubricity between the coating 220 and a surface of a package. Additionally, the lubricant 230 is formulated to provide an effective degree of lubricity between the coating 220 and a coating of another medical device.

Thus, the lubricant 230 helps prevent the medical device 200 from sticking to objects, such as the packaging that contains the medical device or other medical devices that may contact the medical device such as a balloon catheter. Accordingly, as best illustrated in FIG. 2A, the medical device 200 may be at least partially disposed within and may be in contact with a surface of its packaging P and may be moved (i.e., in the direction of arrow A) with respect to the surface of the packaging (i.e. removed from or inserted into the packaging) without causing damage to the medical device 200. Packaging materials with which the lubricant preferably provides sufficient lubricity include, but are not limited to polymer compounds, Tecothane®, and Pebax.

Additionally, the lubricant 230 helps prevent the medical device 200 from sticking to other medical devices during processing. For example, as illustrated in FIG. 2B, the lubricant 230 provides lubrication between the medical device 200 and another medical device 200′ when the medical devices 200 and 200′ are disposed within a packaging P and move with respect to each other (i.e., medical device 200′ is moved in the direction of arrow B). In the illustrated embodiment, the medical device 200′ also includes a lubricant 230′.

In one embodiment, the lubricant 230 provides a mechanical lubrication to the medical device 200. For example, in such an embodiment, the lubricant 230 may be a powder, and the individual particles of the powder may provide a ball-bearing type lubrication. In another embodiment, the lubricant 230 itself may have a slippery property. In yet another embodiment, the lubricant 230 may have a slippery property and provide mechanical lubrication. In another embodiment, the lubricant 230 adheres to the coating 220 and provides a low friction, solid barrier to reduce the stickiness of the surface of the medical device 200.

In the illustrated embodiment, the lubricant 230 is formulated to be soluble in water and in at least one bodily fluid of a mammal, such as blood or urine. Accordingly, the lubricant 230 is formulated to stick to the coating 220 until the medical device is placed in contact with water or the bodily fluid of the mammal. Thus, in one embodiment, the lubricant 230 is formulated to dissolve when the medical device 200 is placed or otherwise disposed within a body of a human patient. In the illustrated embodiment, the lubricant 230 is formulated as a soluble biocompatible powder such as a salt (including potassium chloride) or a sugar (including Mannitol).

In another embodiment, the lubricant 230 may be removed, or partially removed, from the medical device 200 prior to the placement of the medical device 200 in the body of the patient. For example, the lubricant 230 may be washed with water (i.e., the medical device 200 may be dipped in a container of water) prior to placing the medical device 200 within the body of the patient. The water wash may remove all or part of the lubricant 230 from the medical device 200 prior to the placement of the medical device 200 within the body of the patient. If the water wash does not remove all of the lubricant 230 from the medical device 200, the remaining portion of the lubricant 230 may be removed once the medical device is disposed within the body of the patient.

In one embodiment, the coating 220 and coating solvents, such as toluene, tetrahydrofurane, methyl ethyl ketone, chloroform, and/or alcohol, are applied to the medical device 200. The lubricant 230 is then applied to the medical device 200 directly after the coating solvents have been allowed to dry. In another embodiment, the lubricant is applied to the medical device at another time. For example, in one embodiment the lubricant is applied to the medical device at the same time that the coating is being applied.

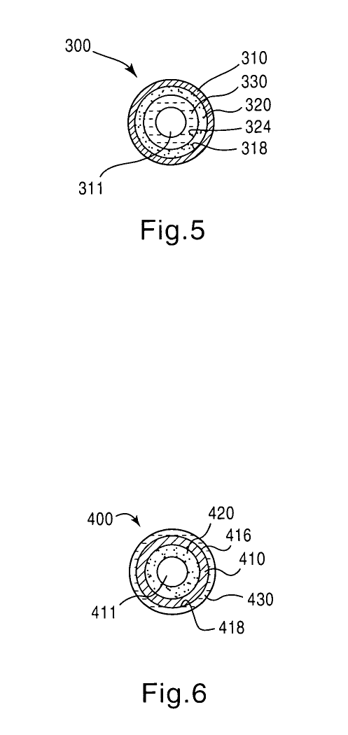

FIG. 5 is a cross-sectional view of another medical device 300. The medical device 300 includes a member 310, a coating 320, and a lubricant 330. The medical device 300 is configured to be placed or otherwise disposed within a body of a mammal or human patient.

As illustrated in FIG. 5, the member 310 defines a lumen 311. The coating 320, including a therapeutic agent, is disposed on an inner surface 318 of the body 310. The lubricant 330 is disposed on an inner surface 324 of the coating 320. In such an embodiment, an object, such as a portion of the member's 310 packaging may be disposed within the lumen 311 of the member 310 without sticking to the inner surface 318 of the member 310.

FIG. 6 is a cross-sectional view of another medical device 400. The medical device 400 includes a member 410, a coating 420, and a lubricant 430. The medical device 400 is configured to be placed or otherwise disposed within a body of a mammal or human patient.

As illustrated in FIG. 6, the member 410 defines a lumen 411. The coating 420, including a therapeutic agent, is disposed on an inner surface 418 of the body 410. The lubricant 430 is disposed on an outer surface 416 of the member 410.

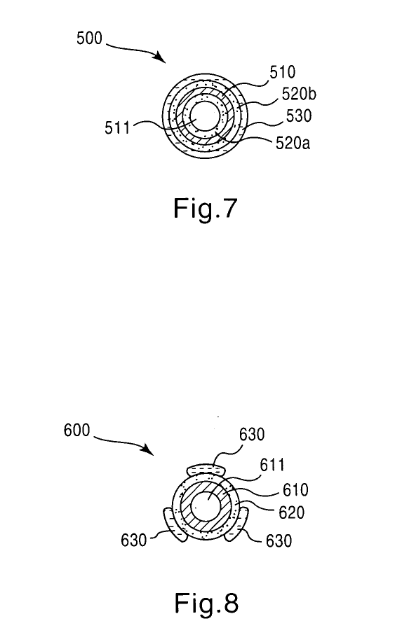

FIG. 7 is a cross-sectional view of another medical device 500. The medical device 500 includes a member 510, a first layer of coating 520a, a second layer of coating 520b, and a lubricant 530. The medical device 500 is configured to be placed or otherwise disposed within a body of a mammal or human patient.

As illustrated in FIG. 7, the member 510 defines a lumen 511. The first layer of coating 520a is disposed on an inner surface of the body 510. The second layer of coating 520b is disposed on an outer surface of the body 510. The lubricant 530 is disposed on an outer surface of the second layer of coating 520b.

FIG. 8 is a cross-sectional view of another medical device 600. The medical device 600 includes a member 610, a coating 620, and a lubricant 630. The medical device 600 is configured to be placed or otherwise disposed within a body of a mammal or human patient.

As illustrated in FIG. 8, the member 610 defines a lumen 611. The coating 620 disposed on an outer surface of the body 610. The lubricant 630 is disposed on a portion, or several portions, of an outer surface of the coating 620.

The principles, preferred embodiments, and modes of operation of the present invention have been described in the foregoing description. However, the invention that is intended to be protected is not to be construed as limited to the particular embodiments disclosed. Further, the embodiments described herein are to be regarded as illustrative rather than restrictive. Variations and changes may be made by others, and equivalents employed, without departing from the spirit of the present invention. Accordingly, it is expressly intended that all such variations, changes and equivalents which fall within the spirit and scope of the present invention as defined in the claims be embraced thereby.

What is claimed is:

1. A medical device for placement within a body of a patient, comprising:
a member;
a coating disposed on at least a portion of the member; and
a lubricant disposed on at least a portion of the coating, the lubricant being formulated to provide an effective degree of lubricity between the coating and at least one of a surface of a package configured to receive at least a portion of the medical device, another portion of the medical device, a coating of another similar medical device, and an uncoated portion of another medical device, the lubricant being formulated to be soluble in at least one of water and a bodily fluid of a mammal.

2. The medical device of claim 1, wherein the coating includes a therapeutic agent.

3. The medical device of claim 1, wherein the coating is formulated to release from the member when the medical device is placed within the body of the patient.

4. The medical device of claim 1, wherein the lubricant is formulated as a powder.

5. The medical device of claim 1, wherein the lubricant is formulated as a biocompatible powder.

6. The medical device of claim 1, wherein the lubricant includes at least one selected from the group consisting of potassium chloride, heparin, Mannitol, and ReoPro®.

7. The medical device of claim 1, wherein the at least one bodily fluid is blood.

8. The medical device of claim 1, wherein the at least one bodily fluid is urine.

9. The medical device of claim 1, wherein the coating includes a polymer.

10. The medical device of claim 1, wherein the body is formed of a metal.

11. An apparatus, comprising:
a member configured to be placed within a body of a patient, the member including a therapeutic agent and a lubricant disposed on a surface of the member, at least a portion of the therapeutic agent being formulated to be released from the member while the member is disposed within the body of the patient, the lubricant being formulated to provide lubrication between the member and one of another such member and a surface of a receptacle configured to receive at least a portion of the member, the lubricant being soluble in at least one of water and a bodily fluid of a mammal.

12. The apparatus of claim 11, wherein the member includes a first end portion and a second end portion, the member defines a lumen that extends from the first end portion to the second end portion.

13. The apparatus of claim 11, wherein the at least a portion of the member defines a lumen, the member includes an inner surface and an outer surface, the lubricant being disposed on at least a portion of at least one of the outer surface and the inner surface of the member.

14. The apparatus of claim 11, wherein the member includes a tubular member.

15. The apparatus of claim 11, wherein the lubricant is formulated as a powder.

16. The medical device of claim 11, wherein the lubricant includes at least one selected from the group consisting of potassium chloride, heparin, Mannitol, and ReoPro®.

17. The apparatus of claim 11, wherein the bodily fluid is blood.

18. A method of packaging a medical device having a coating on at least a portion thereof, the coating including a therapeutic agent, comprising:
disposing a lubricant on at least a portion of the coating;
disposing the medical device at least partially into a package with at least a portion of the lubricant engaging a surface of the package.

19. The method of claim 18, wherein the lubricant is formulated as a powder.

20. The method of claim 18, the medical device being a first medical device, a second medical device having a coating on at least a portion thereof, further comprising:
disposing a lubricant on at least a portion of the coating of the second medical device;
disposing the second medical device at least partially into the package with at least a portion of the lubricant engaging at least one of a surface of the package, the coating of the first medical device, and the lubricant of the first medical device.
